(19) United States
(12) Patent Application Publication
(10) Pub. No.: US 2020/0367790 A1
(43) Pub. Date: Nov. 26, 2020

(54) IMPROVEMENTS IN OR RELATING TO FALL DETECTORS AND FALL DETECTION

(71) Applicant: August International LTD, Hoddesdon Hertfordshire (GB)

(72) Inventors: Keming Zhou, Hoddesdon Hertfordshire (GB); Hongtao Zhang, Hoddesdon Hertfordshire (GB)

(73) Assignee: August International LTD, Hoddesdon Hertfordshire (US)

(21) Appl. No.: 16/764,268

(22) PCT Filed: Nov. 16, 2018
(86) PCT No.: PCT/GB2018/053328

§ 371 Date: May 14, 2020

(30) Foreign Application Priority Data
Nov. 17, 2017 (GB) 1719075.2

(51) Int. Cl. A61B 5/11; G01P 15/18; G01C 19/00; A61B 5/00; A61B 5/0205; A61B 5/145; G08B 21/04

(52) U.S. Cl. CPC A61B 5/1117; A61B 5/021; G01C 19/00; A61B 5/681; A61B 5/7264; A61B 5/746; A61B 5/1118; A61B 5/0205; A61B 5/14542; A61B 5/749; A61B 5/0002; A61B 5/7275; G08B 21/0446; A61B 2562/0219; G01P 15/18

(57) ABSTRACT

A wrist-wearable apparatus for detecting a fall of a wearer, the apparatus includes a device for detecting an acceleration of the apparatus or wearer and determining acceleration magnitude; a device for determining a change in angle of orientation of the apparatus or wearer; a device for detecting and/or determining gyroscope magnitude of the apparatus or wearer; a device for processing acceleration magnitude data and comparing such data with a threshold so as to determine if a potential fall has occurred; and a fuzzy logic device for analysing change in angle of orientation data and gyroscope magnitude data so as to categorise values of such data and, thereby, verify if a fall has occurred.

### CROSS-REFERENCE TO RELATED APPLICATIONS

This application is a U.S. National Phase Application of PCT International Application No. PCT/GB2018/053328, filed Nov. 16, 2018, which claims priority to GB Patent Application No. 1719075.2, filed Nov. 17, 2017, the contents of these applications being incorporated by reference herein in their entirety.

### FIELD OF THE INVENTION

The present invention relates to fall detection and fall detectors. In particular, the present invention relates to a wrist-wearable apparatus for detecting a fall of a wearer and an associated method.

### BACKGROUND OF THE INVENTION

Fall detectors are known in the art and are targeted at the elderly and disabled people, so as to support independent living. Typical prior art detectors are provided to be waist-worn, head-mounted or slung around a chest of a wearer/user. Present fall detectors have various disadvantages, including, that they are expensive to make, difficult to use and difficult to repair. Such fall detectors often suffer from a high-ate of false alarms. Further, such known detectors are also limited in the way they can communicate and cannot send an alarm message automatically, or a user cancel a false alarm.

The present invention is aimed at solving these disadvantages associated with the prior art. In particular, the present invention is aimed at providing a wrist-wearable fall detector which has a low false-alarm rate, and an improved method for verifying if a fall has occurred.

### SUMMARY OF THE INVENTION

According to a first aspect, the present invention provides a wrist-wearable apparatus for detecting a fall of a wearer, the apparatus comprises:
means for detecting an acceleration of the apparatus or wearer and determining acceleration magnitude;
means for determining a change in angle of orientation of the apparatus or wearer;
means for detecting and/or determining gyroscope magnitude of the apparatus or wearer;
means for processing acceleration magnitude data and comparing such data
with a threshold so as to determine if a potential fall has occurred; and wherein the apparatus further comprises fuzzy logic means for analysing change in angle of orientation data and gyroscope magnitude data so as to categorise values of such data and, thereby, verify if a fall has occurred.

Preferably comprising means for processing maximum acceleration magnitude data.

Preferably, the fuzzy logic means is for analysing one or more of the statistics of gyroscope magnitude of the group comprising: maximum; minimum; average; sum; and/or standard deviation.

Preferably, the fuzzy logic means determines an overall fuzzy logic categorisation of low, medium or high for both change in angle of orientation data and gyroscope magnitude data and then:
if overall fuzzy logic output is low or medium, no fall detection alert is triggered; or
if overall fuzzy logic output is high, a fall alert will be triggered.

Preferably, the fuzzy logic means is for additionally analysing one or more of the statistics of acceleration magnitude of the group comprising: maximum; minimum; average; sum; and/or standard deviation.

Preferably, the means for detecting and/or determining gyroscope magnitude is a gyroscope and, most preferably, a three-axis gyroscope.

Preferably, the means for detecting acceleration is an accelerometer and, most preferably a three-axis accelerometer. Most preferably, change in angle of orientation data is derived from the accelerometer.

Alternatively, the apparatus may comprise an inertial measurement unit (IMU), being a combination of accelerometer(s) and gyroscope(s) and, optionally, magnetometer(s).

Preferably, the apparatus further comprises means for detecting a location of said wearer. Preferably, the means for detecting a location of said wearer is a geomagnetic sensor or, preferably, a three-axis geomagnetic sensor or satellite navigation device.

Preferably, additionally comprising means for detecting activity and/or heart rate data. Further preferably, the apparatus comprises means for detecting blood pressure, blood oxygen and/or heart rate data. Preferably, the means is a photoplethysmogram (PPG).

Preferably, the apparatus additionally comprises a voice recognition means for receiving commands from said wearer.

Preferably, the apparatus additionally comprises one or more of the following:
an LED module;
one or more buttons for interaction;
a motor for vibration alerts; and/or
a speaker for audible alerts.

Preferably, the apparatus additionally comprises means for transmitting a fall detection determination and, preferably, automatically.

Preferably, the means for transmitting is configured to use a mobile telephone network and/or short-range wireless technology.

Preferably, the wrist-wearable apparatus is configured so that at least one sensor is capable of contacting the skin of said wearer.

Preferably, a wrist-wearable apparatus comprises a PPG sensor or equivalent, which is configured to contact the skin in the region of a wrist of a wearer, such that heart rate and blood pressure measurements can be taken.

Preferably, the apparatus comprises data collection, processing and transmission means, such that the apparatus is capable of operating independently of a smartphone or computer or the like to detect a fall and issue an alert.

An apparatus for detecting a fall of a wearer, substantially as herein disclosed, with reference to FIG. 1 of the accompanying drawings and/or any example disclosed herein.

According to a second aspect, the present invention provides a method for fall detection, the method comprising:
detecting acceleration of a user and determining acceleration magnitude;
detecting and/or determining change in angle of orientation of said user;
detecting and/or determining gyroscope magnitude of said user;
processing acceleration magnitude data and comparing with a threshold so as
to determine if a potential fall has occurred; and
   wherein the method further comprises using fuzzy logic to analyse change in angle of orientation data and gyroscope magnitude data so as to categorise values of such data and, thereby, verify if a fall has occurred.

Preferably, the method comprises processing maximum acceleration magnitude data so as to determine if a potential fall has occurred.

Preferably, the method comprises analysing one or more of the statistics of gyroscope magnitude of the group comprising: maximum; minimum; average; sum; and/or standard deviation.

Preferably, the method comprises using fuzzy logic to additionally analyse one or more of the statistics of acceleration magnitude of the group comprising: maximum; minimum; average; sum; and/or standard deviation.

Preferably, the method further comprises considering an activity level and/or level of movement of said user after event and, if movement is below a threshold, triggering an alert. Most preferably, using standard deviation of acceleration magnitude data to consider an activity level and/or level of movement of said user.

Preferably, detecting and/or determining gyroscope magnitude using a gyroscope, detecting and/or determining acceleration magnitude using an accelerometer, and/or deriving change in angle of orientation data from the accelerometer.

Preferably, change in angle of orientation and gyroscope magnitude are each categorised as low, medium or high depending upon the data received and analysed, and verifying that a fall has occurred if both are medium or high, or one is medium and the other high.

Further preferably, categorising change in angle of orientation and gyroscope magnitude to provide a value for each between 0 and 100, in which low is 0 to 20; medium is >20 to 60; and high is >60 to 100.

Preferably, collecting real-time acceleration magnitude data and gyroscope magnitude data and, if the acceleration magnitude is greater than a threshold, storing data for subsequent analysis. Further preferably, independently categorising the real-time acceleration magnitude data and gyroscope magnitude data into low, medium and/or high categories, and determining an overall fuzzy logic categorisation of low, medium or high for both change in angle of orientation and gyroscope magnitude and then:
if overall fuzzy logic output is low or medium, no fall detection alert is triggered and reverting back to collecting real-time data again; or
if overall fuzzy logic output is high, collecting further acceleration magnitude data after event.

Most preferably, analysing the after event further acceleration magnitude data and calculating the standard deviation thereof, and then:
if the standard deviation is below a threshold, triggering an alert; or
if the standard deviation is above a threshold, no alert is triggered and reverting back to collecting real-time data again.
   Preferably, the method comprises detecting a location of said user.

Preferably, the method comprises detecting activity, blood pressure and/or heart rate data of said user.

Preferably, the method further comprises triggering sound and/or vibration alerts.

Preferably, the method further comprises transmitting a fall detection determination for the purpose of gaining assistance.

Preferably, the method comprises receiving and acting upon a recognised user's voice commands to raise an alarm or cancel a fall detection determination.

A method for fall detection, substantially as herein disclosed, with reference to FIGS. 2 to 5b of the accompanying drawings and/or any example disclosed herein.

The present invention may also relate to a wrist-wearable apparatus for detecting a fall of a wearer, the apparatus comprises:
means for detecting an acceleration of the apparatus or wearer;
means for detecting an angle of orientation of the apparatus or wearer;
means for processing data relating to acceleration and change in angle of orientation, and comparing such data with one or more thresholds so as to determine if a fall has occurred; and a gyroscope,
   wherein the wrist-wearable apparatus further comprises means for detecting and/or computing acceleration magnitude and fuzzy logic means for analysing change in angle of orientation data and maximum gyroscope magnitude data so as to categorise the value of such data and, thereby, verify if a fall has occurred.

The present invention may also relate to a corresponding method.

Advantageously, the present invention uses a number of sensor inputs and improved ways of analysing and/or processing the data received, so as to reduce the occurrences of false alarms. The algorithm of the present invention is able to filter out a user's normal activities, such as walking, running and sitting, etc. when it is considering whether the sensed data requires the triggering of an alert.

Advantageously, the present invention provides a detector which can issue a warning message containing the user's/wearer's heart rate and location, which can be sent out via short-range wireless technology and/or the mobile network if a fall is detected.

Further advantageously, a user can activate or cancel a warning message through voice control of the fall detector. As a user can activate or cancel a warning message via the voice recognition module, inadvertent triggering of an alert may be avoided. Further, the safety of the user is enhanced through being able to verbally raise an alert.

Further advantageously, a user may cancel a warning message through pressing and holding down a button on the detector apparatus.

Advantageously, the apparatus of the present invention is easy to wear and does not impede the normal activities of a user/wearer. As it is wrist-wearable, the fall detector apparatus is comfortable to wear and more comfortable than traditional waist-worn or chest-slung fall detectors. Warning messages may be sent out via a mobile network or through short-range wireless technology, without the support of a smartphone. Alternatively, by making the short-range wireless technology communication in this fall detector compatible with most smartphones, it is easy for the apparatus to trigger an alert.

An after event, standard deviation of acceleration magnitude threshold is used to prevent false alarms, as people may lay on the ground for a few seconds after a fall.

Further advantageously, the fall detection algorithm of the present invention prevents false alarms even when a user conducts fall-like normal activities such as jumping and clapping.

Further advantageously, using a low-consumption MCU and optimised algorithm enables the apparatus to compute and detect falls independently of a computer or smartphone.

### BRIEF DESCRIPTION OF THE DRAWINGS

The invention will now be disclosed, by way of example only, with reference to the following drawings, in which:

FIG. 1 is a schematic drawing showing the main components of a wrist-wearable fall detector apparatus;

FIG. 2 is a flowchart providing an embodiment of the method for detecting and verifying a fall has occurred;

FIG. 3 is a flowchart providing further details on box 23a of FIG. 2;

FIG. 4 is a flowchart providing further details on box 31 of FIG. 3;

FIGS. 5a and 5b are graphs showing how degree of membership can be calculated for gyroscope magnitude and change in angle of orientation, respectively, before fuzzy logic output is reached.

### DETAILED DESCRIPTION OF PREFERRED EMBODIMENTS

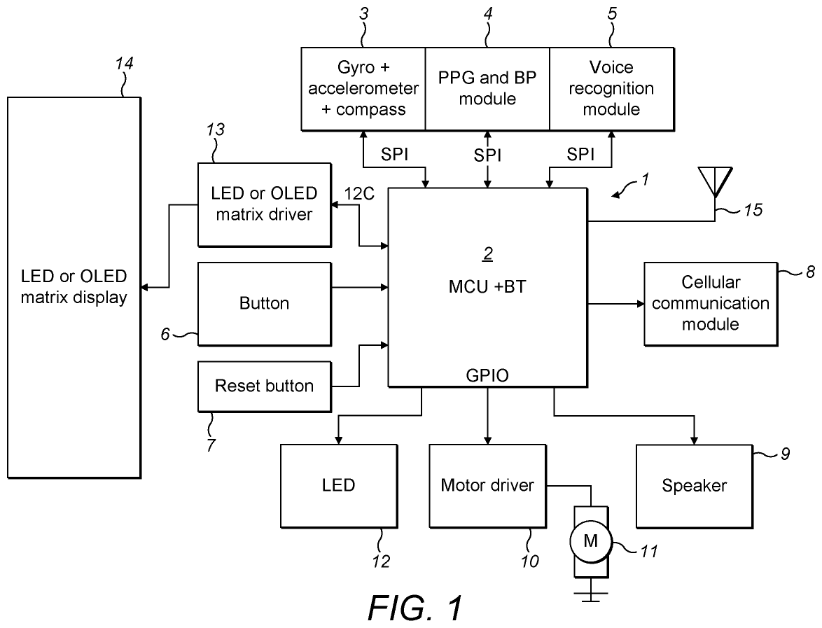

FIG. 1 shows a fall detection apparatus, generally identified by reference 1. The apparatus 1 includes a microcontroller with short-range wireless technology 2 and associated power source (not shown). A number of sensors provide an input to the microcontroller 2, and the apparatus 1 therefore includes a nine-axis sensor 3, being a three-axis gyroscope (providing G_{X}G_{Y}G_{Z}), a three-axis accelerometer (providing A_{X}A_{Y}A_{Z}) and a three-axis geomagnetic sensor (compass), and further a photoplethysmogram and blood pressure module 4 (a PPG sensor). A voice recognition module 5 is also provided as an input to the microcontroller 2, and further inputs are provided by a button 6 and a reset button 7. Outputs from the microcontroller 2 are a cellular communication module 8 (mobile telephone network such as GSM, 3G, 4G, 5G, NB-IoT, etc.) for transmitting through a mobile network, a speaker 9, a motor driver 10 for operating a motor 11, an LED 12 for signalling, and an LED or OLED matrix driver 13 for driving an LED or OLED matrix display 14. An antenna 15 is also provided. The microcontroller 2 acts as the main control chip for the apparatus 1. The LED 12, display 14 and buttons 6; 7 on the apparatus 1 are for user interactions. The motor 11 and speaker 9 are used to raise vibration and voice alarms, respectively.

More specifically, the microcontroller 2 is an MCU+BT having a built in short-range wireless Technology™ module, and is connected with the antenna 15. The microcontroller 2 is connected to the nine-axis sensor 3 with an SDA/SDI interface. Software is pre-installed in the ROM of the microcontroller 2, and data collected from the sensor(s) 3; 4 is/are processed by the microcontroller 2. The microcontroller will send instructions to the imbedded motor 11, speaker 9 and LED/OLED display 14 to raise an alert when a fall has been detected. Further, if a user presses the cancel button or says ‘stop’ within ten seconds of an initial alert—in the latter case control takes place through the voice recognition module 5—and no alarm message will be sent out via short-range wireless technology or a mobile network. In addition, a user may, at any time, say a ‘go’ command to request help if he or she doesn't feel well—again this is implemented through the voice recognition module 5.

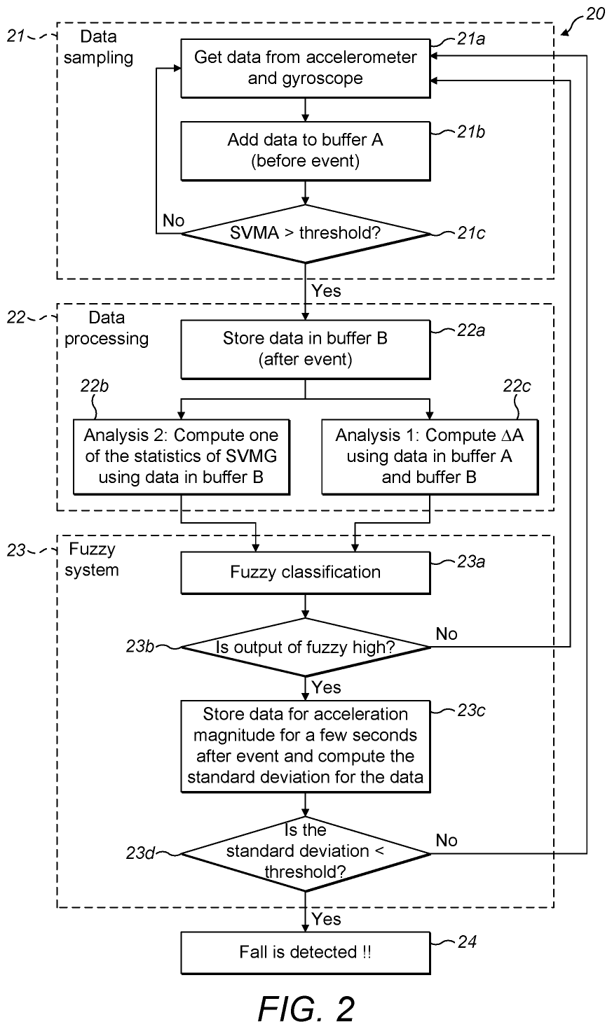

In use, a user wears the apparatus 1 on a wrist and conducts his or her normal daily activities. As the wrist-wearable apparatus 1 is designed to be easily portable, lightweight and unobtrusive, a user can act normally whilst wearing it, in a similar way to wearing a slightly oversized wristwatch. With the apparatus switched on, a user conducting his or her normal activities should not create an alarm signal through carrying out any of those normal activities, as the apparatus is programmed to filter out signals which may trigger prior art fall detectors. The present invention uses a number of sensor inputs and processing of the data received, so as to reduce the occurrences of false alarms, which is elaborated upon in relation to FIG. 2. However, the following provides a simplified version of that procedure.

A data collection block within or associated with the microcontroller 2 collects real-time data from the sensor(s) 3;4. When a magnitude of acceleration from an event exceeds a threshold, a data storage block starts to store data (data before the event and data after the event). The data is then transferred to a data analysis block when the data storage block is full. Data from two inputs, which data is change in angle of orientation and the maximum gyroscope magnitude data, is sent to a fuzzy logic system to analyse the possibility of a fall. If the output of the fuzzy logic system is low or medium, the algorithm will go back to the start and collect new data. However, if the output of the fuzzy logic system is high, data after the fall event is collected and the standard deviation of that data is calculated. If the standard deviation is over a threshold, then the algorithm will go back to the start and collect new data. However, if the standard deviation is below a threshold, a fall alert will be triggered. This standard deviation threshold is used to prevent false alarms, as people may lay on the ground for a few seconds after a fall.

FIG. 2 shows a flowchart 20 providing a graphical representation of an algorithm which is operated by the microcontroller 2 of the fall detection apparatus 1. The flowchart 20 may be split into three regions of operation, a first region 21 being data sampling, a second region 22 being data processing, and a third region 23 being fuzzy system.

With respect to the data sampling region 21, real-time data before an event is retrieved from the accelerometer and the gyroscope—box 21a—and added to Buffer A—box 21b—which stores 1,500 data, for example. The data obtained is three-axis data for accelerations A_{X}A_{Y}A_{Z} and angular velocities G_{X}G_{Y}G_{Z}. Once the data has been added to Buffer A and, after an event, the data is compared with a threshold—box 21c—and if the acceleration magnitude is greater than the threshold, then the fall detection method continues into data processing 22. However, if the acceleration magnitude is less than the threshold, then the algorithm goes back to collecting real-time data, as per box 21a.

With respect to data processing 22, data received from data sampling 21 is stored in Buffer B—box 22a—which stores 1,500 data, for example. Two forms of analysis are conducted under data processing 22. One form of analysis computes the maximum gyroscope magnitude using the data in buffer B—as per box 22b. The other form of analysis involves computing the change in angle of the device using data in Buffers A and B—as per box 22c which uses the equation ΔA defined at the end of the description. The outputs from boxes 22b and 22c are fed into the fuzzy system 23.

As for the fuzzy system 23, this uses the data from boxes 22b and 22c to determine an overall fuzzy classification, the fuzzy output of which can be low, medium or high—as per box 23a. Once a fuzzy output has been determined, actions are assigned depending upon the determination, as per box 23b. If the fuzzy output is high, then store for example 1,000 data for the acceleration magnitude for a few seconds after the event and compute the standard deviation for that data, as per box 23c. However, if the output is not high (i.e. is low or medium), then the algorithm goes back to collecting real-time data, as per box 21a. Following a high fuzzy output, that standard deviation of acceleration magnitude is compared with a threshold, as per box 23d. This part of the algorithm provides a check to see what has happened after the fall event, and considers the level of movement and/or level of activity of the user. For instance, if a user has fallen, one would expect a period of relative inactivity even after a minor fall or prolonged inactivity after a major fall which then reinforces the fall verification. If the standard deviation is below a given threshold, then positive detection of a fall has been achieved, as indicated in box 24. However, if the standard deviation is not below a given threshold, then the algorithm goes back to collecting real-time data, as per box 21a.

With respect to a system which does not use fuzzy logic, if the algorithm has two inputs of change in angle and gyroscope magnitude which are scored from 0 to 100, and the thresholds of both inputs are set at 50, without fuzzy logic a system will not detect a fall unless both inputs are over 50. So, if one input is 49 and the other is 99, a fall alert will not be triggered. However, through using fuzzy logic, one is able to detect falls at the fringes of fall conditions.

Accordingly, in a first embodiment, a simplified determination of fuzzy classification according to box 23a can be conducted as follows. The two inputs of change in angle and maximum gyroscope magnitude are initially categorised as low, medium or high, according to the following Table 1.

TABLE 1
Initial Fuzzy Categorisation.

| Label | Low(L) | Medium(M) | High(H) |
|---|---|---|---|
| Input1 (Angle) | 0-20 | >20-60 | >60-100 |
| Input2 (Gyroscope) | 0-20 | >20-60 | >60-100 |

A decision matrix is then created for an overall fuzzy logic output, which depends upon the categorisation of the two inputs in Table 1. The decision matrix is Table 2 below.

TABLE 2
Decision Matrix

| Output | Input 1 = L | Input 1 = M | Input 1 = H |
|---|---|---|---|
| Input2 = L | L | M | M |
| Input2 = M | M | H | H |
| Input2 = H | M | H | H |

By way of example, according to the decision matrix above, if input 1 is medium and input 2 is medium, then the overall fuzzy logic output is high, and a fall alert will be triggered as it has been verified by the fuzzy logic. In essence, if input 1 and 2 are either medium or high, that will lead to an overall fuzzy logic output of high.

Those skilled in the art will understand that Tables 1 and 2 provide a simple example of the kind of fuzzy logic proposed by the Applicant; however, there could be more inputs, and the categorisation of the inputs and the decision matrix itself could be more complex.

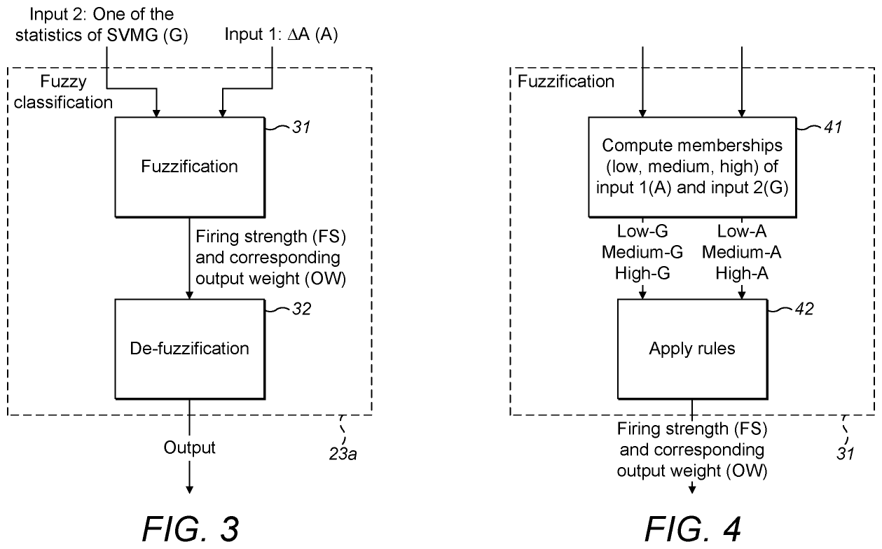

By way of an alternative embodiment, in a second embodiment a more in-depth determination of fuzzy classification according to box 23a is described in relation to FIGS. 3, 4 and 5a and b, and can be conducted as follows.

As shown in FIGS. 3 and 4, fuzzy classification 23a involves two processes: fuzzification 31; and de-fuzzification 32.

Fuzzification 31 itself involves two processes being: computing memberships 41 and applying rules 42.

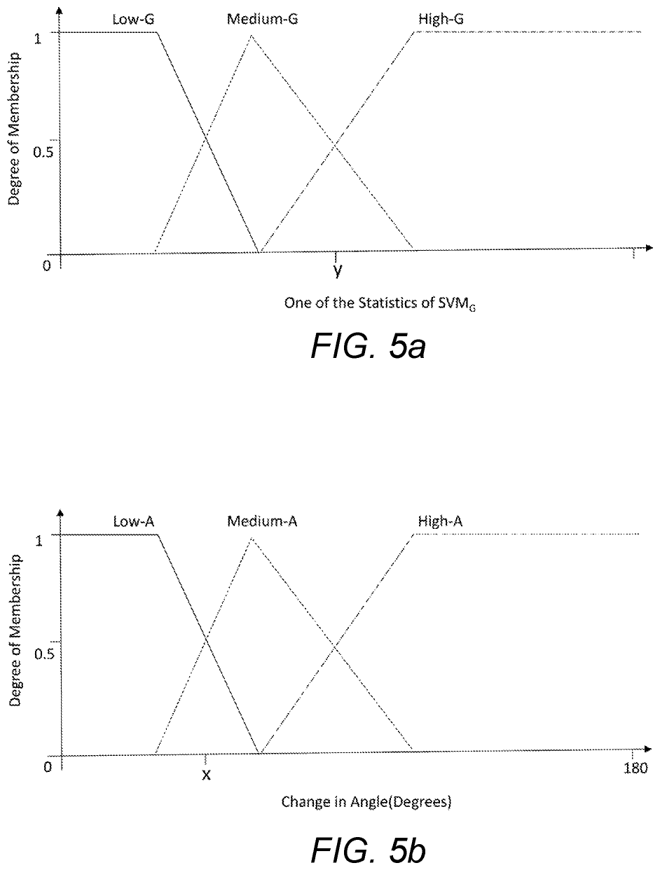

In this particular example, computing memberships 41 involves the use of graphs 5a and 5b, which define a numerical degree of membership for a data point for gyroscope magnitude (G) and change in angle of orientation (A) in each of the three categories of low, medium and high. Using FIG. 5a, for a data point Y, low-G=0, medium-G=0.5, and high-G=0.5, and, using FIG. 5b, for a data point X, low-A=0.5, medium-A=0.5, and high-A=0—which provides an overall six memberships.

Once the memberships have been calculated, the rules are applied and Table 3 provides exemplary rules.

TABLE 3
Rules

| | | | | Output | Firing |
|---|---|---|---|---|---|
| Number | Input 1: | Input 2: | | Weight | Strength |
| of rule | ΔA | SVM_{G} | Output | (OW) | (FS) |
| Rule 1 | LOW | LOW | LOW | 10 | FS1 |
| Rule 2 | LOW | MEDIUM | LOW | 10 | FS2 |
| Rule 3 | LOW | HIGH | LOW | 10 | FS3 |
| Rule 4 | MEDIUM | LOW | LOW | 10 | FS4 |
| Rule 5 | MEDIUM | MEDIUM | MEDIUM | 30 | FS5 |
| Rule 6 | MEDIUM | HIGH | HIGH | 50 | FS6 |
| Rule 7 | HIGH | LOW | LOW | 10 | FS7 |
| Rule 8 | HIGH | MEDIUM | MEDIUM | 30 | FS8 |
| Rule 9 | HIGH | HIGH | HIGH | 50 | FS9 |

According to Table 3, nine rules apply to the six memberships, which then provide nine corresponding output weights (OW) and nine corresponding firing strengths (FS).

By way of explanation, the OW of LOW in the fourth column has been set as 10 (but it could be between 0 and 20), the OW of MEDIUM has been set as 30 (but it could be between 20 and 40), and the OW of HIGH has been set as 50 (but it could be between 40 and 60).

By way of further explanation, FS can be calculated from using, for example, an average, maximum, minimum, or sum value of the two degrees of membership for A and G (as calculated from FIGS. 5a and 5b). Thereby, using a minimum for rule 7, if high-A=0.4, low-G=0.5, then FS7=Minimum of (0.4 and 0.5)=0.4.

De-fuzzification 32 is now possible, and such is achieved through use of the following:

$$Output = \frac{{\sum }_{i = 1}^{9} ( {FS}_{i} * {OW}_{i} )}{{\sum }_{i = 1}^{9} {FS}_{i}}$$

According to this equation an overall fuzzy output is determine—which is the final stage of box 23a. The fuzzy output is then assessed in box 23b, as described above.

Those skilled in the art will understand that linear relationships are shown in FIGS. 5a and 5b; however, those relationships are exemplary and do not, in practice, have to be linear. FIGS. 5a and 5b show these relationships graphically from which manual calculations can be taken for ease of reference; however, in practice they are likely to be calculated automatically. In addition, the rules and thereby the outputs are exemplary and, therefore, some deviation from those shown could be used.

The following definitions and equations are provided for the avoidance of doubt and so as to provide a reference for the skilled person.

Acceleration Magnitude means sum vector magnitude of acceleration (SVM_{A}), which is provided by:

SVM_{A}=\sqrt{Aₓ²+A_{y}²+A_{z}²}

Accordingly, maximum acceleration magnitude is the maximum value of SVM_{A}.

Gyroscope Magnitude means sum vector magnitude of gyroscope (SVM_{G}), which is provided by:

SVM_{G}=\sqrt{Gₓ²+G_{y}²+G_{z}²}

Accordingly, Maximum gyroscope magnitude is the maximum value of SVM_{G}.

Change in Angle of Orientation means change in angle of the device (ΔA) from the start to the end of a fall event. It is calculated from data obtained from the accelerometer according to the equation below in which Aₓₛ and A_{xE} means acceleration of the x-axis at the start and at the end of the fall event, respectively, and so on for the y- and z-axes. For clarity, data at the start of the event is derived from Buffer A data and data at the end of the event is derived from Buffer B data.

$$Δ A = {cos}^{- 1} ( \frac{( {A}_{xs} * {A}_{xE} ) + ( {A}_{ys} * {A}_{yE} ) + ( {A}_{zs} * {A}_{zE} )}{\sqrt{( {A}_{xs}^{2} + {A}_{ys}^{2} + {A}_{zs}^{2} ) * ( {A}_{xE}^{2} + {A}_{yE}^{2} + {A}_{yE}^{2} )}} )$$

1.-34. (canceled)

35. A wrist-wearable apparatus for detecting a fall of a wearer of the wrist-wearable apparatus, the wrist-wearable apparatus comprises:
means for detecting an acceleration of the apparatus or the wearer and determining acceleration magnitude;
means for determining a change in an angle of orientation of the apparatus or the wearer;
means for detecting and/or determining a gyroscope magnitude of the apparatus or the wearer;
means for processing acceleration magnitude data and comparing said acceleration magnitude data with a threshold so as to determine if a potential fall has occurred; and
fuzzy logic means for analyzing (i) change in angle of orientation data and (ii) gyroscope magnitude data so as to categorize values of such data and, thereby, verify if a fall has occurred.

36. The apparatus as claimed in claim 35, wherein the fuzzy logic means is configured for analyzing one or more of the statistics of a gyroscope magnitude of a group comprising: maximum; minimum; average; sum; and/or standard deviation.

37. The apparatus as claimed in claim 35, wherein the fuzzy logic means is configured to determine an overall fuzzy logic categorization of low, medium or high for changes in the angle of orientation data and the gyroscope magnitude data and then:
if overall fuzzy logic output is low or medium, no fall detection alert is triggered; or
if overall fuzzy logic output is high, a fall alert will be triggered.

38. The apparatus as claimed in claim 35, wherein the fuzzy logic means is configured for additionally analyzing one or more of the statistics of an acceleration magnitude of a group comprising: maximum; minimum; average; sum; and/or standard deviation.

39. The apparatus as claimed in claim 35 further comprising means for detecting a location of said wearer, activity, blood pressure, blood oxygen and/or heart rate data.

40. The apparatus as claimed in claim 35 further comprising a voice recognition means for receiving commands from said wearer.

41. The apparatus as claimed in claim 35 further comprising means for transmitting a fall detection determination.

42. The apparatus as claimed in claim 35 in which the apparatus comprises data collection, processing and transmission means, such that the apparatus is capable of operating independently of either a smartphone or a computer to detect a fall and issue an alert.

43. A method for fall detection comprising:
detecting acceleration of a user and determining an acceleration magnitude;
detecting and/or determining a change in angle of orientation of the user;
detecting and/or determining a gyroscope magnitude of the user;
processing acceleration magnitude data and comparing the acceleration magnitude data with a threshold so as to determine if a potential fall has occurred; and
analyzing change in angle of orientation data and gyroscope magnitude data using fuzzy logic so as to categorize values of such data and, thereby, verify if a fall has occurred.

44. The method as claimed in claim 43 comprising processing maximum acceleration magnitude data so as to determine if a potential fall has occurred.

45. The method as claimed in claim 43, wherein the method comprises analyzing one or more of the statistics of gyroscope magnitude of a group comprising: maximum; minimum; average; sum; and/or standard deviation.

46. The method as claimed in claim 43 comprising using fuzzy logic to additionally analyze one or more of the statistics of an acceleration magnitude of a group comprising: maximum; minimum; average; sum; and/or standard deviation.

47. The method as claimed in claim 43, wherein the method further comprises considering an activity level and/or level of movement of said user after an event and, if movement is below a threshold, triggering an alert.

48. The method as claimed in claim 43, wherein a change in angle of orientation and gyroscope magnitude are each categorized as low, medium or high depending upon data received and analyzed, and verifying that a fall has occurred if both the angle of orientation and gyroscope magnitude are medium or high, or one of angle of orientation and gyroscope magnitude is medium and the other of angle of orientation and gyroscope magnitude is high.

49. The method as claimed in claim 43 comprising collecting real-time acceleration magnitude data and gyroscope magnitude data and, if the acceleration magnitude is greater than a threshold, the method comprises storing data for subsequent analysis.

50. The method as claimed in claim 49 further comprising independently categorizing the real-time acceleration magnitude data and gyroscope magnitude data into low, medium and/or high categories, and determining an overall fuzzy logic categorization of low, medium or high for both change in angle of orientation and gyroscope magnitude and then:
if overall fuzzy logic output is low or medium, no fall detection alert is triggered and the method reverts back to collecting real-time data again; or
if overall fuzzy logic output is high, the method further comprises collecting further acceleration magnitude data after an event.

51. The method as claimed in claim 50 further comprising analyzing the after-event further acceleration magnitude data and calculating a standard deviation thereof, and then:
if the standard deviation is below a threshold, the method comprises triggering an alert; or
if the standard deviation is above a threshold, no alert is triggered and the method reverts back to collecting real-time data again.

52. The method as claimed in claim 43 comprising detecting a location of the user, activity, blood pressure and/or heart rate data of the user.

53. The method as claimed in claim 43 further comprising transmitting a fall detection determination for the purpose of gaining assistance.

54. The method as claimed in claim 43 comprising receiving and acting upon a recognized user's voice commands to raise an alert or cancel a fall detection determination.
